(19) United States
(12) Patent Application Publication
(10) Pub. No.: US 2013/0281956 A1
(43) Pub. Date: Oct. 24, 2013

(54) BIOHAZARD CONTAINMENT GARMENT

(71) Applicants: Michael Kevin Mulcahey, Acton, CA (US); Duane Bronk, Tujunga, CA (US)

(72) Inventors: Michael Kevin Mulcahey, Acton, CA (US); Duane Bronk, Tujunga, CA (US)

(21) Appl. No.: 13/867,938

(22) Filed: Apr. 22, 2013

Related U.S. Application Data
(60) Provisional application No. 61/636,079, filed on Apr. 20, 2012.

(51) Int. Cl. A62B 17/00; A61F 13/496

(52) U.S. Cl. CPC A62B 17/006; A61F 13/496
USPC 604/385.13; 2/79; 604/385.01

(57) ABSTRACT

A garment for containing fluids includes: a trouser portion comprising integral foot coverings and a fluid impermeable material; and an upper body portion integrated with the trouser portion.

### CROSS-REFERENCE TO RELATED APPLICATION

This application claims the benefit of U.S. Provisional Patent Application No. 61/636,079, titled “Biohazard Containment Garment,” filed in the United States Patent and Trademark Office on Apr. 20, 2012, the entirety of which is incorporated by reference herein.

### BACKGROUND

In various working environments such as medical facilities, penal facilities, and law enforcement settings, there is a risk that uncooperative or mentally unstable individuals can (intentionally or unintentionally) spread hazardous materials such as urine, fecal matter, blood, blood borne pathogens, saliva, and vomit within those working environments. Such contamination can pose serious health risks for people working in those environments and often results in significant cleanup costs.

### SUMMARY

There is a demand for devices and methods for containing hazardous materials (such as bodily fluids) in order to prevent such materials from contaminating a working environment.

Embodiments of the present invention are directed to disposable biohazard containment suits/garments to protect the wearer's custodian or the public in general from various hazards and/or to protect the workplace from contamination by the persons wearing the biohazard containment suit. Such a disposable containment garment may be designed for single-use, after which they may be discarded.

For example, in a law enforcement setting, such a garment would facilitate the careful handling and transport of ill, inebriated, or injured persons when it is necessary to take such persons into custody and would offer the advantage of substantially eliminating the expensive need to clean a contaminated police/transport vehicle if hazardous materials were excreted by such persons.

Furthermore, such disposable biohazard containment suits/garments can be used to contain biological evidence within the garment. For example, blood samples on the suspect may be contained by the garment (and not spread across the environment) and after the garment is removed at the police station, the garment itself can be entered into evidence.

Embodiments of the present invention relate to biohazard containment suits/garments which include both a trouser portion with integral foot coverings and an upper body portion. In some embodiments, the containment suit/garments are designed to be discarded after they become contaminated.

According to one embodiment of the present invention, a garment for containing fluids, the garment includes: a trouser portion comprising integral foot coverings and a fluid impermeable material; and an upper body portion integrated with the trouser portion.

The trouser portion may include an absorbent material.

The absorbent material may be located between a waistline portion of the trouser portion and a thigh portion of the trouser portion.

The absorbent material may be located in the integral foot coverings.

The upper body portion may include a breathable material.

The garment may further include a hook and loop fastener adapted to seal a front access opening of the garment.

The garment may further include a hook and loop fastener adapted to seal a rear access opening of the garment.

The garment may further comprises a hook and loop fastener adapted to seal an arm opening of the garment.

The garment may further include a band adapted to encircle the upper body portion, the strap comprising a hook and loop fastener.

The garment may further include a rear control handle coupled to the band.

The may further include a receptacle, the receptacle including: a tapered bag; a splash shield; an internal fluid absorption panel; and a hook and loop fastener, wherein the hook and loop fastener of the receptacle is attached to the hook and loop fastener of the strap.

The garment may further include a suspension system, the suspension system including: a cinch point adapted to control the tension of the suspension system; angle garter attachment points coupled to the integral foot coverings; a support sling coupled to a crotch portion of the trouser portion; and shoulder loops coupled to the upper body portion.

The suspension system may be formed of webbing.

### BRIEF DESCRIPTION OF THE DRAWINGS

The accompanying drawings, together with the specification, illustrate exemplary embodiments of the present invention, and, together with the description, serve to explain the principles of the present invention.

FIG. 1 is a schematic front view of a containment suit/garment in accordance with one embodiment of the present invention.

FIG. 2 is a schematic rear view of a containment suit/garment in accordance with one embodiment of the present invention.

FIG. 3 is a schematic view of absorption layers in a trouser portion of a containment suit/garment in accordance with one embodiment of the present invention.

FIG. 4 is a schematic side view of a containment suit illustrating a suspension system in accordance with one embodiment of the present invention.

FIG. 5 is a schematic perspective view of a suspension system in accordance with one embodiment of the present invention.

FIG. 6 is a schematic views of a vomit containment receptacle according to one embodiment of the present invention.

### DETAILED DESCRIPTION

The accompanying drawings, together with the specification, illustrate exemplary embodiments of the present invention, and, together with the description, serve to explain the principles of the present invention. Like reference numerals designate like elements throughout the specification.

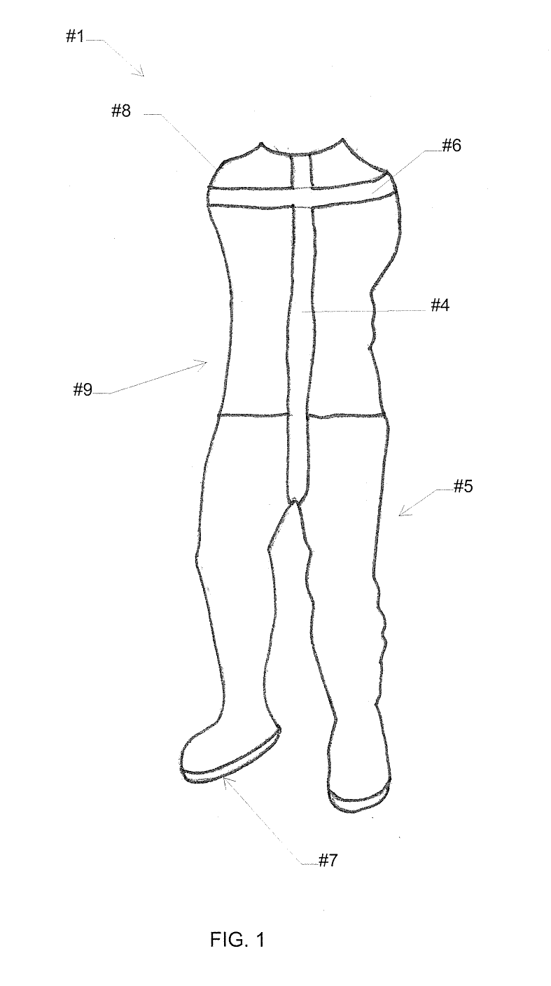

FIG. 1 is a schematic front view of a containment suit/garment in accordance with one embodiment of the present invention. A one-piece protective suit #1 has a trouser (or pant or pants) portion #5 attached to an upper body portion #9. The suit has a front access opening, indicated diagrammatically by the central line composed of hook and loop type fastening material #4 (e.g., Velcro®).

Referring to FIG. 1, the trouser portion #5 with integral foot enclosures (or coverings) #7 of the suit #1 and a trouser portion #5 of the upper body portion #9 may be formed from a first material and the upper part #9 of the upper body portion may be formed from (or include) a second material different from the first material. According to one embodiment, the foot enclosures #7 are configured to enclose the feet of the wearer and integrated with the portion. In addition, the trouser portion #5 and the upper body portion #9 may be integrated together.

In this specification, when two components are referred to as being “integrated” or “integral” then the two components may be formed as the same continuous piece of material, the two portions may be welded together, or otherwise attached such that there is a seal which substantially prevents the leakage of fluid at a junction between the two components.

In one embodiment, the first material employed for the trouser portion #5 of the suit is a barrier material that is impervious (or impermeable) to fluids while the second material employed for the upper part #9 is a breathable barrier material.

The trouser portion #5 of the suit #1 may be formed of a fabric or other suitably flexible barrier material that is substantially impervious (or impermeable) to fluids (e.g., bodily fluids). Suitable materials include closed plastic films (e.g., polyolefin films) conventionally employed for protective disposable clothing, laminates thereof, and injection molded one piece constructions. In some embodiments, the material is capable of being welded to form sealed seams.

The second material may be any of a number of breathable materials such as high-density polyethylene fabric that are well known to those of ordinary skill in the art. Any breathable material which is both suitable for forming into garments and capable of containing the fluids likely to be encountered during use of the suit may be used for the upper part #9 of the suit #1.

Embodiments of the present invention may be constructed out of a low cost material that is consistent with the degree of containment desired in the intended usage scenario. For example, plastic films are attractive materials from both their cost point and their impermeability to fluids and may offer a high degree of protection against biologically hazardous or contaminated liquids.

In some embodiments of the present invention, the upper body portion #9 is sleeveless and lacks openings for arms for use with individuals who have their arms restrained (e.g., handcuffed) behind their backs. In other embodiments, the upper area of the suit/garment where sleeves would normally be attached includes openings #8 which may be closed with a hook and loop type fastener. This may be opened if it is desired that the wearer remain in the suit/garment without their arms being restrained. In some embodiments, the openings #8 may span from the shoulder area to the waistline area of the suit/garment.

The suit #1 shown in FIG. 1 may be used in circumstances in which there is a risk of the wearer's body contaminating surrounding surfaces, vehicles, and personnel.

Generally, lining the trouser portion #5 of the suit (and not the upper body portion #9) with a barrier material that is impervious to fluid would be sufficient to contain bodily fluids because, generally, discharge of urine and fecal matter occurs below the waistline. In those cases, the effective degree of protection offered to the custodian of the wearer is generally only slightly reduced, if at all, by forming the upper body portions #9 of the suit #1 without including a barrier material that is impervious to fluid.

In some embodiments of the present invention, the comfort of the suit may be increased substantially when the upper body portion is not formed of a material that is impervious to fluids because this allows the passage of air and water vapor (e.g., sweat) from the wearer's body through the upper parts #9 of the suit, thereby reducing the risk of overheating. The build-up of heat inside a containment suit is an important issue, not only from the point of view of comfort but also from the point of view of safety. Generally, if the core body temperature of the wearer rises by more than about 1° C., he/she is at risk from heat stress which is dangerous and can even result in death.

Provided that the breathable part of the suit constitutes a large enough part of the total surface area of the suit, wearing the suit should not significantly increase the risk of heat stress, thereby enabling the suit to be worn for extended periods of time. In some embodiments, the breathable part of the suit/garment constitutes at least 40% of the total surface area of the suit and, in other embodiment, about 69% of the total surface area of the suit. Heat loss through the upper portion of the body is generally more effective in preventing a rise in core body temperature than heat loss through other parts of the body such as the legs.

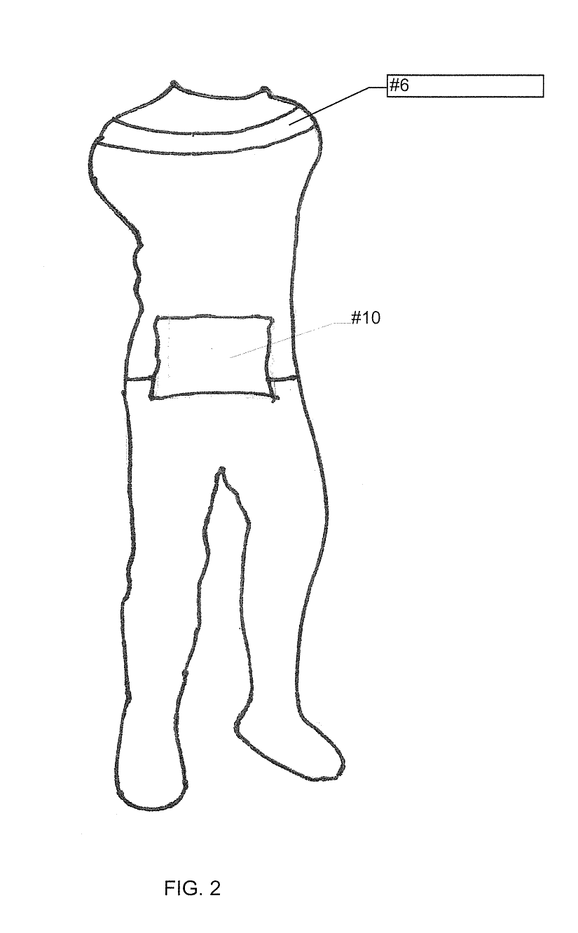

Referring to FIG. 2, in some embodiments, the suit includes an open-able flap #10 secured with hook and loop type fastening materials in the rear of the trouser portion, above the waistline of the suit/garment #1. This flap #10 may be used to allow inspection of the handcuffs and hands to ensure, for example, that the wearer's hands are properly secured such that circulation to the wearer's hands is not being cut off.

In some embodiments, a portion of the upper body portion #9 and/or the trouser portion #5 is formed of a transparent material (e.g., a transparent plastic) in an area to allow inspection of restrained arms and hands (e.g., the hands of a person wearing handcuffs). For example, the transparent portion may extend from a seat portion of the trouser portion #5 to a lower back portion of the upper body portion #5 (e.g., in a portion corresponding to the open-able flap #10 shown in FIG. 2).

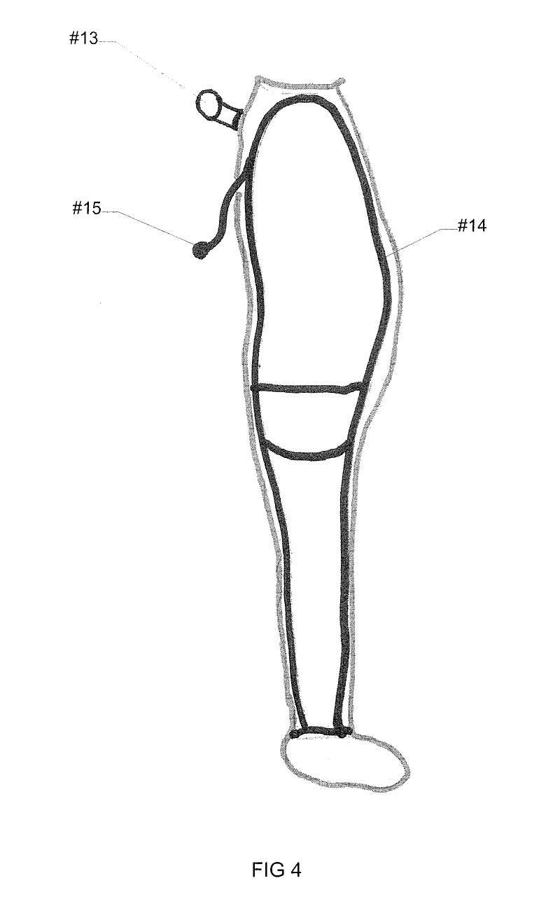

Referring to FIGS. 1 and 2, according to one embodiment, the suit/garment includes a wide (e.g., 2″) band #6 of hook and loop fastener material spanning from shoulder to shoulder across the front and back upper portion of the suit/garment. The band #6 may be split at the main entry point of the suit garment and continue around to the back to form a complete circle (e.g., to encircle the upper portion #9. This swath of hook and loop material will perform two functions: 1) to spread the structural load of the rear control handle #13 (see FIG. 4) affixed to the upper back portion of the suit/garment; and 2) to provide an additional flap of double sided hook and loop fasting material to be secured across the chest area to prevent opening of the main entry point of the suit/garment when force is applied to the rear control handle #13.

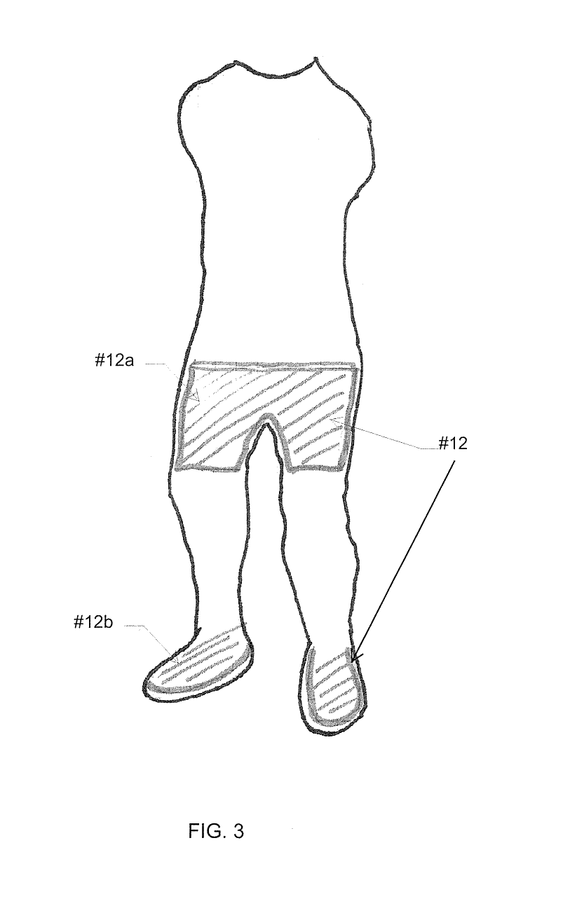

The protection offered by a suit/garment according to embodiments of the present invention as shown, for example, in FIGS. 1, 2, and 3 can be further enhanced by reducing the number of potential leakage points in the suit and by reducing any leakage that does occur. The potential leakage points in a containment suit include: the seams generally; at or below the waistline; ankles; around the foot enclosure; and at the access opening (e.g. the front opening #4). Additional structures for reducing leakage at those points are described below.

Various methods are available for reducing leakage at the seams of containment garments. For example, U.S. Pat. No. 4,683,593 describes the use of ultrasonic welding to form the seams in a suit which is made of a plastic film laminate while U.S. Pat. No. 4,593,418 describes the inclusion, in a stitched seam, of an elastomeric tape. It is also known that covering stitched seams with tape can reduce leakage.

In some embodiments, welding (e.g., ultrasonic welding) may be used to achieve seams which have a high integrity. Consequently, in such embodiments, the materials used to form the garment should be capable of being welded (including to each other in the case of the suit #1 shown in FIGS. 1, 2, and 3). For example, polypropylene is a material that is suitable for ultrasonic welding. In the case of the suit #1 according to embodiments illustrated in FIGS. 1, 2, and 3, the fluid impermeable material may be a polypropylene or polyethylene film and the breathable barrier material may be a polypropylene spun-melt-spun (SMS) material, enabling all of the seams in and between those materials to be ultrasonically welded.

As a further aid to reducing leakage at the seams in a containment garment, in some embodiments of the present invention an absorbent inner liner #12 is provided in those parts of the garment that are likely to be subject to a higher degree of fluid or human waste matter discharge when the garment is being worn, thereby reducing the stress on the seams in those areas. For example, in the case of containment suits of the type shown in FIGS. 1, 2, and 3, a liner of absorbent material can be incorporated into the suit from the waistline to the upper thigh areas #12a. Additional absorbent material #12b may be incorporated into the foot enclosures in order to absorb any fluids that bypass the upper absorbent area or that are compressed (e.g., squeezed) out of the upper absorbing area by movement or sitting.

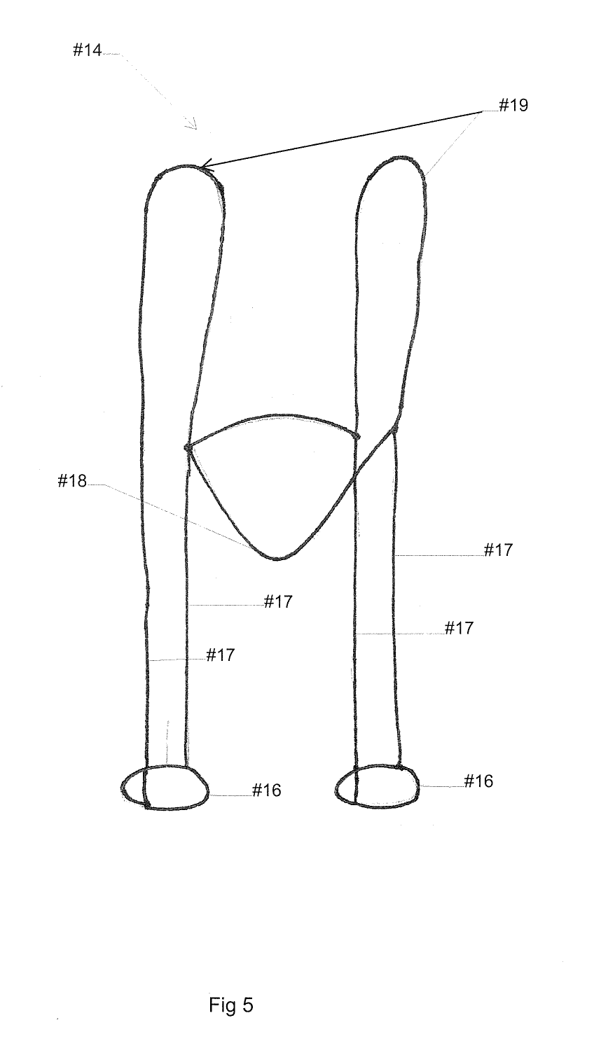

Referring to FIGS. 4 and 5, in some embodiments, the suit #1 further includes a suspension system #14. The suspension system #14 may be located between the layers of the containment suit #1 as depicted in FIGS. 4 and 5. The suspension system #14 may include a cinch point #15 which may be used to control and adjust the tension of the suspension system #14, ankle garter attachment points #16 to allow the foot covering portion to be pulled tight against the toes of the wearer, vertical support risers #17 to allow the wearer's shoulders to bear the load of pulling the garment up and tight against the toes and crotch area to reduce trip hazards, support sling webbing #18 attached to the underside of the absorbent material to secure the crotch of the suit and absorbent material against the wearer to improve absorption, and shoulder loops #19 to transfer the load on the lower straps (e.g., the vertical support risers) to the shoulders of the wearer. The cinch point #15 is configured to allow the custodian of the wearer to adjust the containment suit in such a fashion to pull the excess material from the foot covering area to the tip of the toes as well as the crotch material of the containment suit up securely against the crotch of the wearer. By making these adjustments, ease of movement by the wearer will be improved, thereby reducing the risk of trip/fall by the wearer and increasing the effectiveness of the absorption lining. Adjustments to the suspension system may be made by pulling on one or more points or straps in the suspension system #14.

According to one embodiment of the present invention, the suspension system #14 includes 1″ wide webbing (e.g., nylon webbing) secured within the shell of the suit. The bearing load of suit adjustment is placed upon the shoulders of the wearer. As illustrated in FIGS. 4 and 5, according to one embodiment, the webbing runs from the shoulders down to the crotch and ankle area connecting to attachment points in those areas respectively.

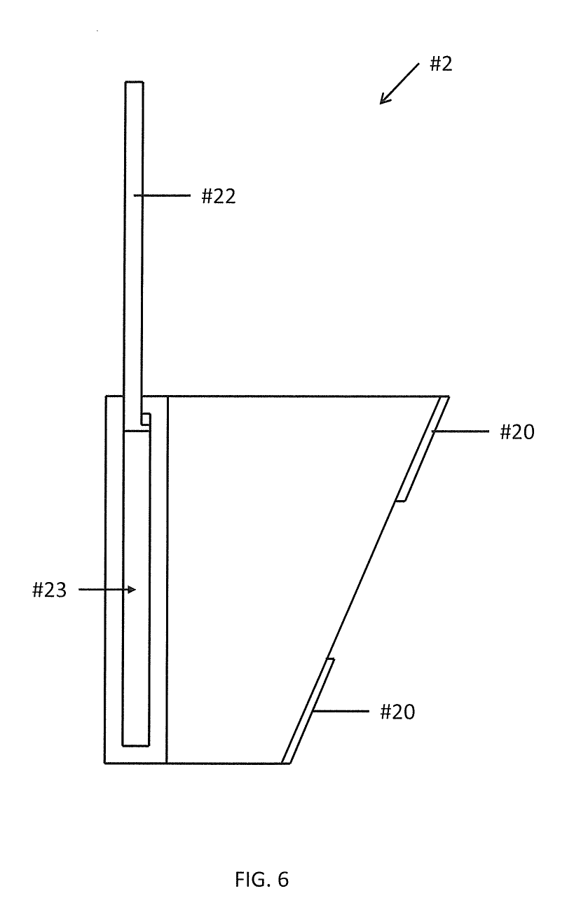

Referring to FIG. 6, in some embodiments of the present invention a detachable vomit collection receptacle #2 includes of a tapered bag type structure with splash/diverter shield #22 and internal fluid absorption panels. The detachable vomit collection receptacle #2 may include a hook and loop fastening strip #20, which may be used to affix the detachable vomit collection receptacle #2 to the double sided hook and loop fastening strip #6 on the front side of the upper body portion #9 of the suit #1. In some embodiments, the splash/diverter shield #22 is configured to telescope (e.g., be retractable and extendable) into a cavity #23 in the vomit collection receptacle #2. The vomit collection receptacle #2 may be formed of substantially the same material as the trouser portion #5.

While the present invention has been described in connection with certain exemplary embodiments, it is to be understood that the invention is not limited to the disclosed embodiments, but, on the contrary, is intended to cover various modifications and equivalent arrangements.

What is claimed is:

1. A garment for containing fluids, the garment comprising:
a trouser portion comprising integral foot coverings and a fluid impermeable material; and
an upper body portion integrated with the trouser portion.

2. The garment of claim 1, wherein the trouser portion comprises an absorbent material.

3. The garment of claim 2, wherein the absorbent material is located between a waistline portion of the trouser portion and a thigh portion of the trouser portion.

4. The garment of claim 2, wherein the absorbent material is located in the integral foot coverings.

5. The garment of claim 1, wherein the upper body portion comprises a breathable material.

6. The garment of claim 1, wherein the garment further comprises a hook and loop fastener adapted to seal a front access opening of the garment.

7. The garment of claim 1, wherein the garment further comprises a hook and loop fastener adapted to seal a rear access opening of the garment.

8. The garment of claim 1, wherein the garment further comprises a hook and loop fastener adapted to seal an arm opening of the garment.

9. The garment of claim 1, wherein the garment further comprises a band adapted to encircle the upper body portion, the band comprising a hook and loop fastener.

10. The garment of claim 9, further comprising a rear control handle coupled to the band.

11. The garment of claim 9, further comprising a receptacle, the receptacle comprising:
a tapered bag;
a splash shield;
an internal fluid absorption panel; and
a hook and loop fastener,
wherein the hook and loop fastener of the receptacle is attached to the hook and loop fastener of the band.

12. The garment of claim 1, further comprising a suspension system, the suspension system comprising:
a cinch point adapted to control the tension of the suspension system;
angle garter attachment points coupled to the integral foot coverings;
a support sling coupled to a crotch portion of the trouser portion; and
shoulder loops coupled to the upper body portion.

13. The garment of claim 12, wherein the suspension system is formed of webbing.
